(19) United States
(12) Patent Application Publication
(10) Pub. No.: US 2024/0148540 A1
(43) Pub. Date: May 9, 2024

(54) COLLAPSIBLE CONTINGENCY URINAL

(71) Applicant: IRPI LLC, Wilsonville, OR (US)

(72) Inventors: Ryan M. Jenson, Happy Valley, OR (US); Oleg Krishcko, Vancouver, WA (US); Logan Torres, Hillsboro, OR (US); Mark Weislogel, Newberg, OR (US)

(21) Appl. No.: 18/503,021

(22) Filed: Nov. 6, 2023

Related U.S. Application Data
(60) Provisional application No. 63/382,607, filed on Nov. 7, 2022.

(51) Int. Cl. A61F 5/451; A61F 5/44; A61F 5/441

(52) U.S. Cl. CPC A61F 5/451; A61F 5/4404; A61F 5/441

(57) ABSTRACT

Methods and systems are provided for a liquid capture device, comprising a superhydrophobic expansion chamber, a user interface coupled to a first end of the superhydrophobic expansion chamber; and a collapsible bag coupled to a second end of the superhydrophobic expansion chamber, opposite the first end. The liquid capture device may be used to capture urine in reduced gravity environments.

### CROSS REFERENCE TO RELATED APPLICATIONS

The present application claims priority to U.S. Provisional Application No. 63/382,607, entitled “COLLAPSIBLE CONTINGENCY URINAL”, and filed on Nov. 7, 2023. The entire contents of the above-listed application are hereby incorporated by reference for all purposes.

### FIELD

The present description relates generally to capture and direction of liquid using superhydrophobic and superhydrophilic materials.

### BACKGROUND/SUMMARY

Hygienic urine collection is difficult in microgravity as liquids in space are dominated by capillary forces and are difficult to manage. Under nominal circumstances, spacecraft toilets use airflow to pull the urine away from the body and into the collection system. Urine collection is crucial for crew hygiene, and insufficient urine collection can be life-threatening. If the toilet is non-functional or inaccessible, a contingency device is desired which may contain urine in a passive manner for long-duration missions such as those to the moon. In a contingency scenario where toilet airflow is not available, managing urine becomes difficult as liquid stays attached to the body at low flow rates and may break up into challenging-to-manage droplet sprays at high flow rates. It is imperative for crew hygiene and health that urine be contained. Attachment of urine to the body may be a more common challenge for female users, due to the nature of female anatomy. Thus, a device for wicking urine away from a user's body is desired which may further enable hygienic removal of urine and may be especially useful in reduced or low-gravity environments. A passive device is ideal for a contingency scenario as it may not use volatile resources, like power, to function. Superhydrophobic and superhydrophilic surfaces are shown to be effective as a passive method of dictating the orientation, movement, and stability of fluids in microgravity. This, coupled with geometric features, can be applied to passive urine management.

Previous contingency devices include the Urine Collection Device (UCD) and Maximum Absorbency Garment (MAG). The UCD was a single-use device that included an interface for females, and a condom catheter for males. The interface was connected to a bag that started out in the deflated state and was inflated by the urination process, removing use of venting internal air. The MAG is a garment worn by astronauts that operate similarly to a diaper. MAGs are currently used during liftoff, extra-vehicular activities, and landing. One of the limitations of the UCD device was that it imposed back pressure on the user which may not allow for complete void and may have contributed to urinary tract infections. Additionally, the urine holding bag was constructed of heat-sealed plastic which proved to be leaky. Finally, the UCD was a single-use device, which meant each astronaut used a new UCD for each urination, raising volume and mass concerns. The MAG is also a single-use device and would use significantly more mass and volume as a long-duration flight contingency. Astronauts have also shown a dislike for the use of MAG devices.

According to embodiments of the present disclosure, microgravity urine collection devices were developed that are hygienic, collapsible, and reusable for the accommodation of astronauts as a contingency measure aboard spacecraft. A collapsible contingency urinal (CCU) device takes advantage of superhydrophobic and superhydrophilic materials as well as CCU device geometry to control the flow regime, trajectory, orientation, and stability of urine in microgravity environments. The CCU described herein uses a novel expanding cusp geometry that is lined with superhydrophobic surfaces to facilitate the hygienic separation of fluid from the body and expulsion downstream into the bag, axially. The CCU also uses a superhydrophobic screen to allow for air displacement from the bag, removing back-pressure on the user. The CCU uses superhydrophobic surfaces throughout to eliminate the buildup of urine where it is not desired (e.g., a lid), as well as superhydrophilic vane and outer bag structure to drive liquid to a drain port, separate gas bubbles at the surface, and increase its stability for handling and draining operations.

In one embodiment, a urine capture device comprises a superhydrophobic expansion chamber, a user interface coupled to a first end of the superhydrophobic expansion chamber; and a collapsible bag coupled to a second end of the superhydrophobic expansion chamber, opposite the first end. The superhydrophobic expansion chamber is configured as a liquid self-propelling passage, comprising a superhydrophobic passage configured to enable liquid self-propelling away from a first end of the superhydrophobic passage towards a second end of the superhydrophobic passage, opposite the first end, wherein the superhydrophobic passage has a first cross-sectional width at the first end and a second cross-sectional width at the second end, the second cross-sectional width greater than the first cross-sectional width, such that the superhydrophobic passage has a bell shape that flares from the first cross-sectional width to the second cross-sectional width.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an example of a collapsible contingency urinal (CCU) with and without removable elements coupled thereto.

FIG. 2 shows an exploded view of the CCU of FIG. 1.

FIG. 3A shows a side view, a top view, and a cross-sectional profile view of the CCU of FIG. 1.

FIG. 3B shows an annotated profile view of the CCU of FIG. 1.

FIG. 4 shows perspective views of a user interface which may be coupled to the CCU of FIG. 1.

FIG. 5 shows an internal configuration of a liquid self-propelling passage, and a housing therefore, which may be included in the CCU of FIG. 1.

FIG. 6 shows a development of liquid flow through the liquid self-propelling passage of FIG. 5.

FIG. 7 shows a venting ring of the CCU of FIG. 1 coupled to a superhydrophobic expansion chamber, which includes the liquid self-propelling passage of FIGS. 5 and 6.

FIG. 8 shows an exploded view of a collapsible bag of the CCU of FIG. 1.

FIG. 9 shows an exploded view of vane structures of the CCU of FIG. 1.

FIG. 10 shows a perspective view and a profile view of the vane structures of FIG. 9.

FIG. 11 shows a plurality of collapsed CCUs, which are examples of the CCU of FIG. 1.

FIG. 12 illustrates a method for directing liquid flow through the CCU of FIG. 1.

### DETAILED DESCRIPTION

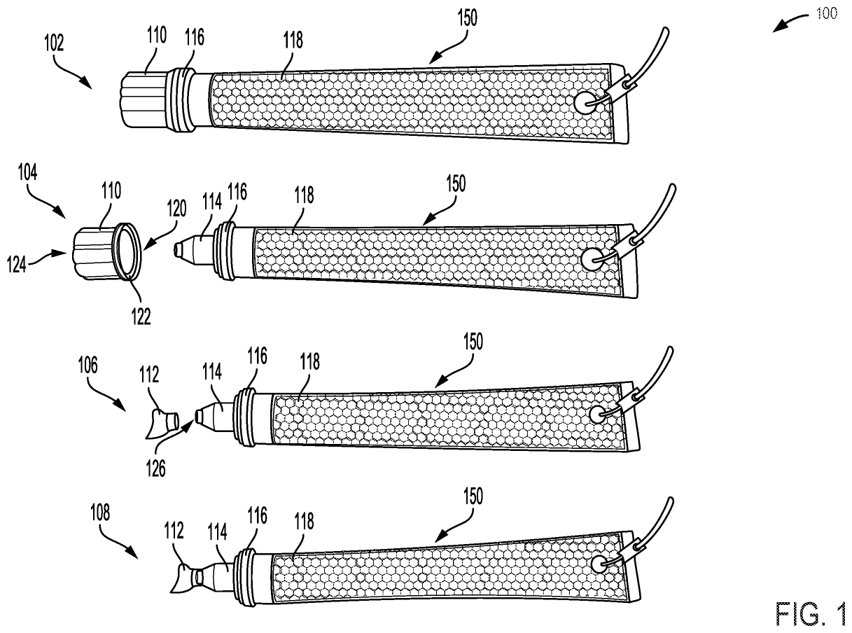

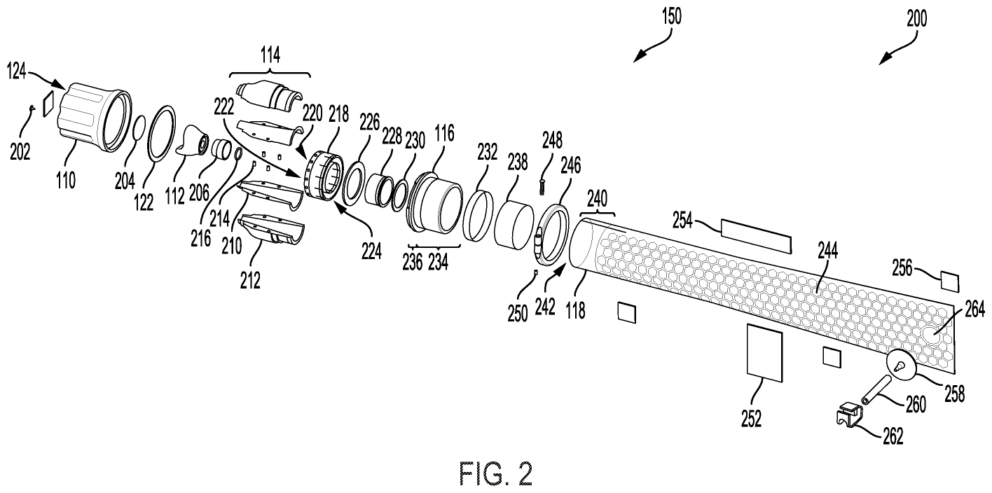

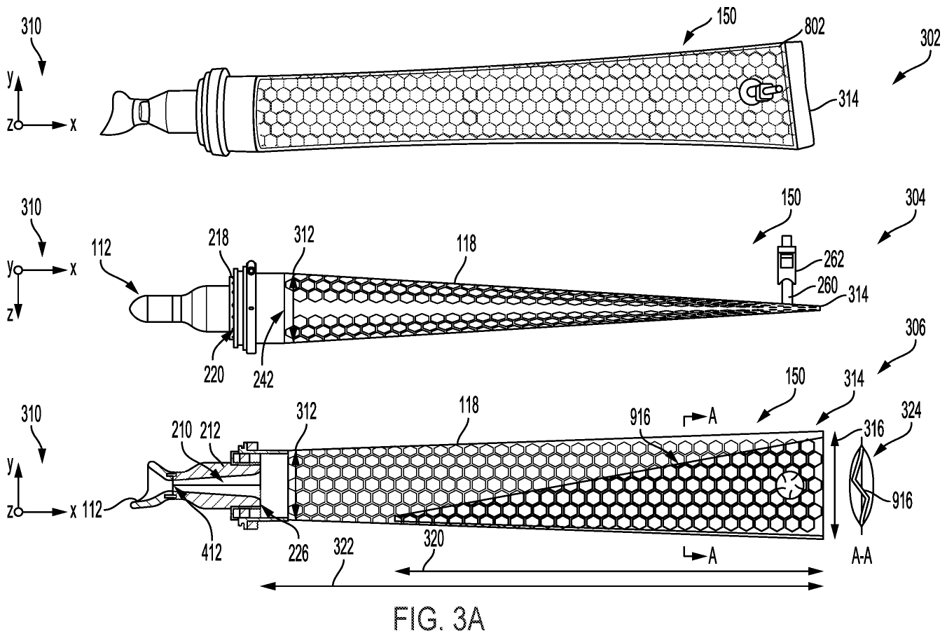

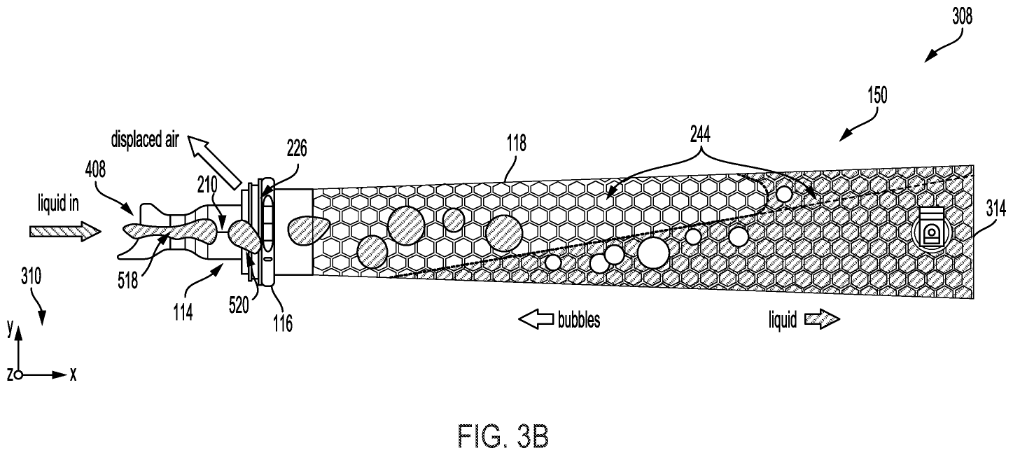

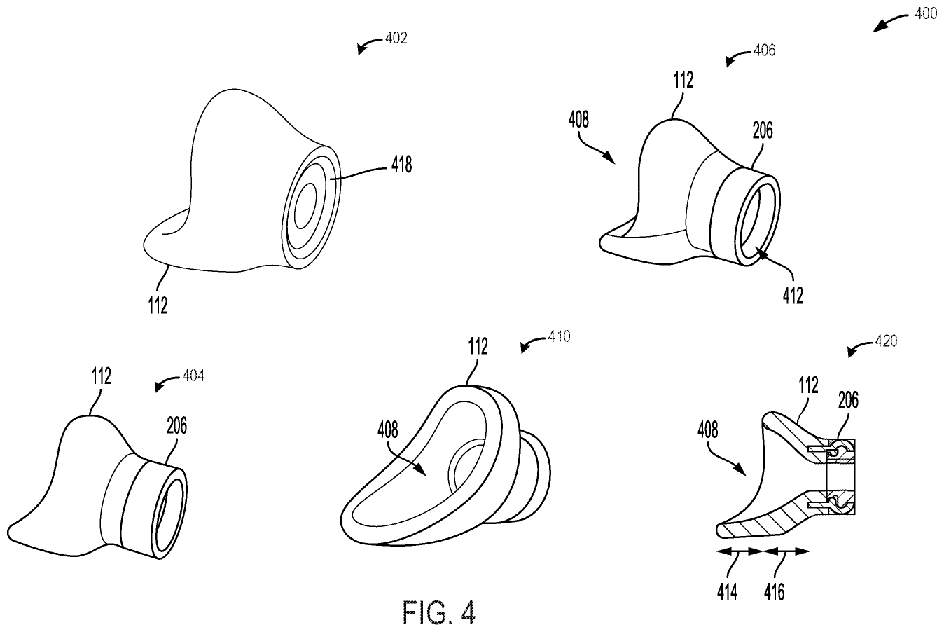

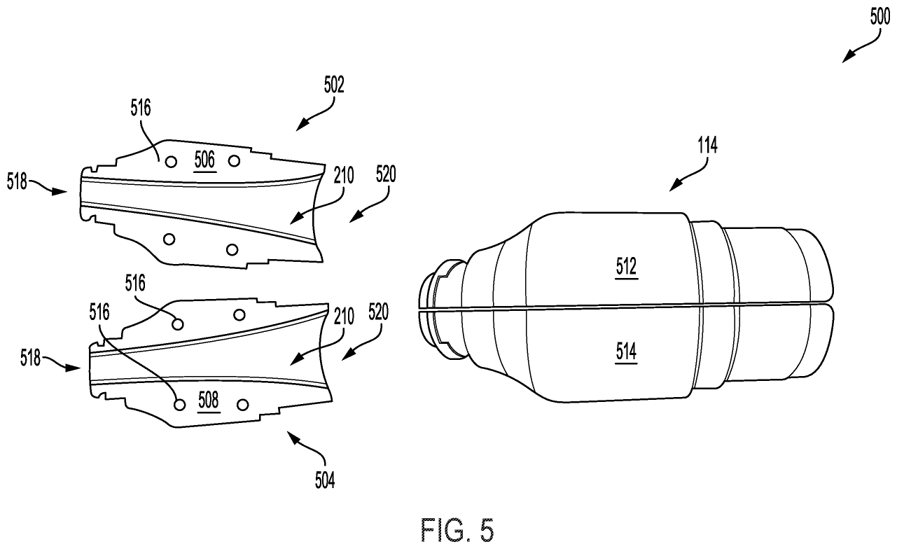

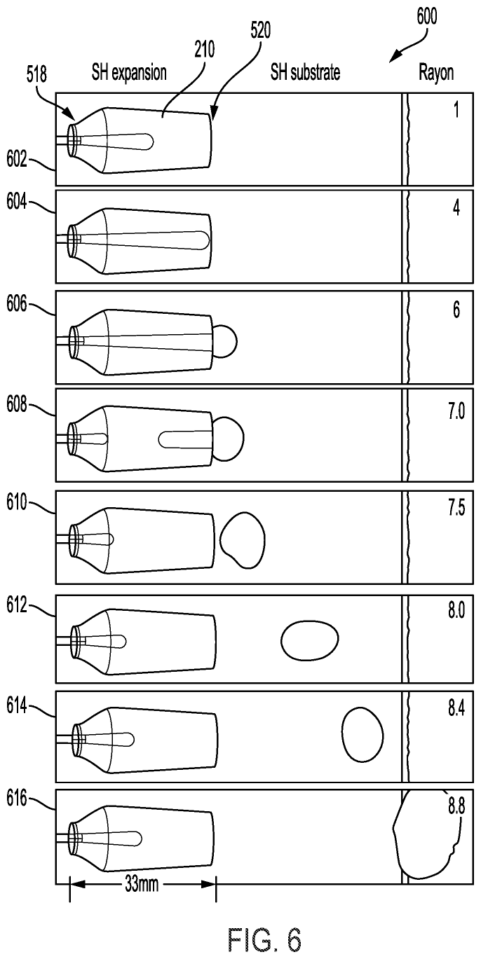

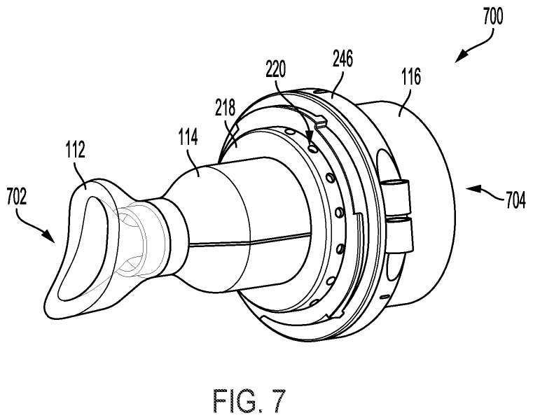

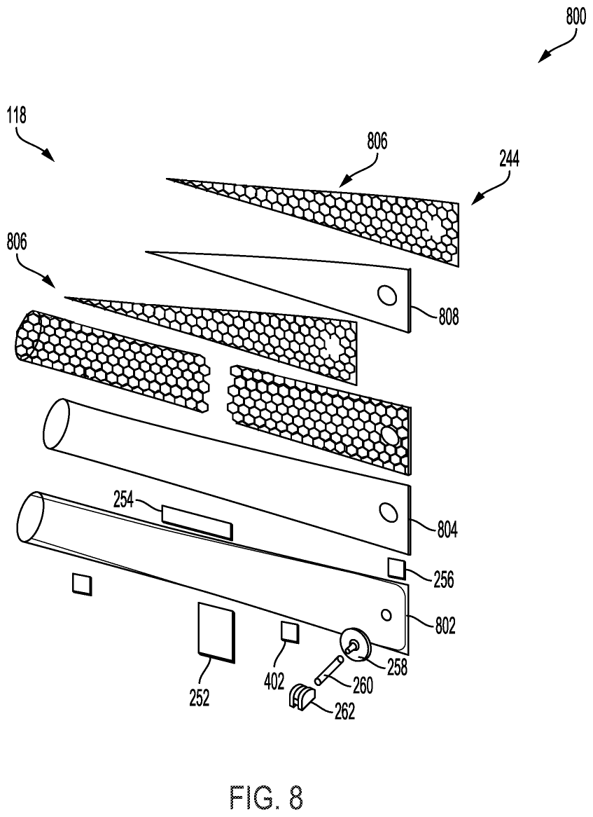

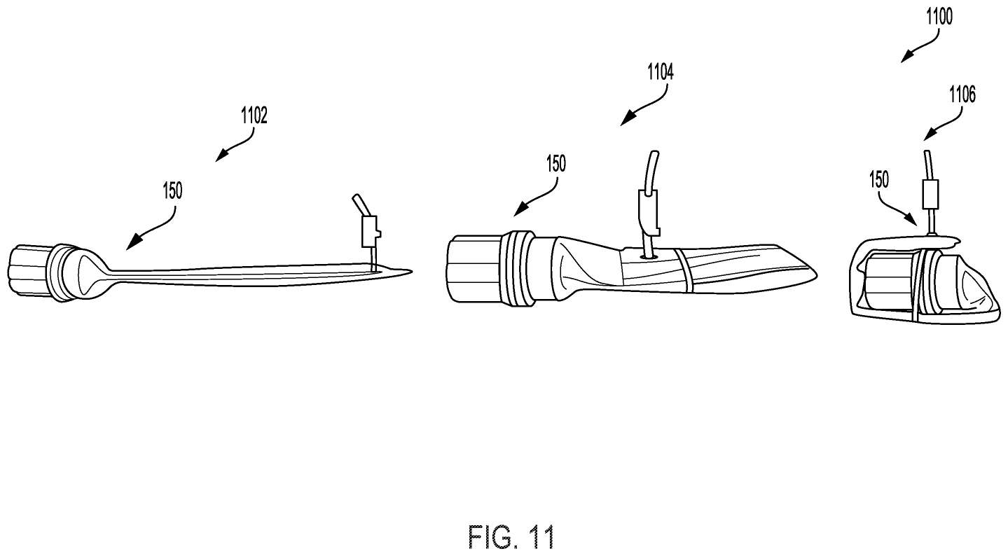

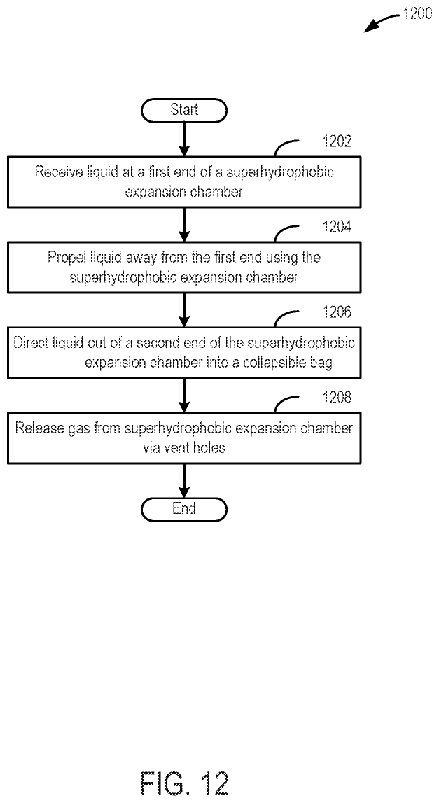

The following description relates to systems and methods for a collapsible contingency urinal (CCU). Elements of the CCU use superhydrophobic and superhydrophilic materials to direct liquid flow in the CCU. FIG. 1 shows an embodiment of the CCU with and without a plurality of removable elements coupled thereto. An exploded view of elements of the CCU is shown in FIG. 2. The CCU is configured with superhydrophobic elements and superhydrophilic elements to facilitate self-propelling of liquid in a first direction and displacement of gas in a second direction, opposite the first direction. FIG. 3A shows a plurality of views of the CCU and FIG. 3B shows an annotated profile view of the CCU, indicating flow of liquid and gas in the CCU. Briefly, liquid may be disposed into the CCU via a user interface, as described with respect to FIG. 4. The user interface may be coupled to a superhydrophobic expansion chamber including a superhydrophobic passage, as further described with respect to FIG. 5, which enables liquid self-propelling away from a first end of the superhydrophobic passage towards a second end of the superhydrophobic passage, opposite the first end, as further described with respect to FIG. 6. The superhydrophobic expansion chamber may be coupled to a liquid capture system, herein a collapsible bag, of the CCU by a vent ring, as described with respect to FIG. 7. The collapsible bag is configured with superhydrophilic elements including a vane structure and a bag liner, as further described with respect to FIGS. 8-10. The CCU may be collapsed by being folded, rolled, or laid flat, as shown in FIG. 11. A method for flowing liquid through the CCU using elements described herein is illustrated in FIG. 12. FIGS. 1-11 are shown approximately to scale, though other scaling may be used without departing from the scope of the present disclosure.

The routine hygienic collection and processing of urine aboard spacecraft remains difficult. This is perhaps as attributable to the myriad resources used for spaceflight life support as it is to the acute challenges of multiphase fluid physics in microgravity. The CCU device described herein exploits recent advances in microgravity capillary fluidics research, combining robust superhydrophobic and superhydrophilic substrates with container geometry that mimic gravity, where, in effect, droplets ‘fall’ and bubbles ‘rise’.

The CCU design described herein allows in part for simple, clean, single- and multiple-void contributions providing local storage, accurate volumetric measures of urine contributed (in the manner of a graduated cylinder on Earth), vent to space or processing equipment, and service as contingency in the event that a main toilet is unavailable (e.g., non-functional and/or inaccessible). Despite the banality of toilet systems on Earth, the weightless environment aboard orbit or coast spacecraft poses several significant challenges that have been known to the aerospace community. Conventional toilet systems used aboard spacecraft and in space suffer a variety of nuisances including un-optimized ergonomics, hygienics, backflows, re-use limitations, and mass penalties. For example, such a device may be designed to account for the low-g passive control of a perturbed transitional motive liquid jet that breaks up into a droplet-laden two-phase flow. The droplet-laden two-phase flow may ingest bubbles when the bubbles come in contact with the liquid jet within the device, which has poorly wetting characteristics for liquids, such as urine. Such fluid physics occurs on Earth, but is easily and entirely accommodated by the dominating ever-present acceleration of gravity—fill from the ‘top’ and drain from the ‘bottom’—while droplets fall and bubbles rise. A device is desired which provides a method to passively receive and reject liquid (e.g., urine away from a body) using a collapsible collection container that passively coalesces jets and droplets which come in contact with elements of the collection container, wicks the liquid toward the exit port, and drives gas bubbles to merge with a free surface (e.g., an interface between the liquid and air in the device), and a screen filter that allows displaced air and not liquid to escape during the process.

FIG. 1 shows an example of a CCU 150 with and without removable elements coupled thereto. Briefly, the CCU 150 is configured with a lid 110, a user interface 112, a superhydrophobic expansion chamber 114, an outer snap ring 116, and a collapsible bag 118 including superhydrophilic elements. Briefly, all references to superhydrophobic surfaces and/or elements imply substrates with a liquid contact angle θ>150° with low hysteresis (e.g., less than ±5°). For example, a superhydrophobic surface and/or element having the above described properties may be formed of a non-wetting material (e.g., having a liquid contact angle θ>90°), such as laser ablated polytetrafluoroethylene (PTFE) and/or fluorinated ethylene propylene (FEP), laser ablated silicone, and/or a coating or a variety of methods used to impart superhydrophobic properties on a material used to form the referenced element. Superhydrophilic surfaces may include any wetting material, e.g., liquid contact angle θ=0° with no hysteresis, such as Rayon/Polyester and/or any variety of absorbent material such as Rayon and a blend/carrier, such as Polyester.

A first configuration 102 shows the CCU 150 with the lid 110 coupled to the outer snap ring 116. A second configuration 104 shows the CCU 150 with the lid 110 uncoupled from the outer snap ring 116. A third configuration 106 shows the CCU 150 with the user interface 112 uncoupled from the superhydrophobic expansion chamber 114. A fourth configuration 108 shows the CCU 150 with the user interface 112 coupled to the superhydrophobic expansion chamber 114.

Removable elements of the CCU 150 include elements which may be removed or attached prior to and/or following use of the CCU 150, and include the lid 110 and the user interface 112. The lid 110 may be selectively attached to the CCU 150, for example, by screwing in or snap fitting the lid 110 to the outer snap ring 116. When the lid 110 is coupled to the outer snap ring 116 by screwing the lid 110 to the outer snap ring 116, each of the lid 110 and the outer snap ring 116 may be configured with threading to enable coupling thereof. For example, the lid 110 may be configured with threading at an opening 120 of the lid 110. When the lid 110 is coupled to the outer snap ring 116 by snap fitting, the lid 110 may include a lip at the opening 120, and the outer snap ring 116 may be configured with a complimentary radial extension which may fit inside then lip of the lid 110 when force is applied to the lid 110 to snap fit the lid 110 and the outer snap ring 116 together. Further details regarding the outer snap ring 116 are provided with respect to FIGS. 2 and 7. The lid 110 may further comprise a gasket 122 positioned at the opening 120 of the lid 110, such that when the lid 110 is coupled to the outer snap ring 116, the lid 110 may fluidically seal an interior of the collapsible bag 118 of the CCU 150 from an external environment, which may prevent liquid from leaking out of the CCU 150 as further described herein. The gasket 122 may have an adhesive backing to couple the gasket 122 to the lid 110. Additionally, the lid 110 may be configured with an umbrella valve (described with respect to FIG. 2) at a top 124 of the lid 110, opposite the opening 120 of the lid 110, which may allow gas to escape from the CCU 150, via the lid 110, when the lid is coupled to the CCU 150 at the outer snap ring 116. For example, the umbrella valve may enable pressure relief for the CCU 150, such as relieving pressure from the collapsible bag 118 to reduce pressurization of the collapsible bag 118. Additionally or alternatively, when the CCU 150 is used in a cabin of a vehicle in space, the umbrella valve may assist in pressure relief of the CCU 150 during cabin depressurization.

When the lid 110 is removed from the CCU 150 (e.g., detached from the outer snap ring 116), the user interface 112 may be coupled to the superhydrophobic expansion chamber 114 at a first end 126 of the superhydrophobic expansion chamber 114. Further detail regarding the user interface 112 is described with respect to FIGS. 3A-4 and further detail regarding the superhydrophobic expansion chamber 114 is described with respect to FIGS. 3A, 3B, 5, and 6.

Turning to FIG. 2, an exploded view 200 of the CCU 150 is shown. Elements of the CCU 150 are introduced herein with respect to FIG. 2 and will be elaborated on with respect to subsequent figures. Elements of the CCU 150 described with respect to FIG. 1 are equivalently numbered in FIG. 2 and subsequent figures. As described with respect to FIG. 1, the CCU 150 includes the lid 110, the user interface 112, the superhydrophobic expansion chamber 114, the outer snap ring 116, and the collapsible bag 118. The lid 110 is configured with an umbrella valve 202, as well as a lid liner 204 and the gasket 122. The umbrella valve 202 may extend through the top 124 of the lid 110 and may be at least partially secured in place by the lid liner 204. The CCU 150 further includes the user interface 112 which may be selectively coupled to the CCU 150. For example, the user interface 112 may be coupled to the superhydrophobic expansion chamber 114 of the CCU 150 via a quarter-turn adapter 206, where the user interface 112 is configured with an annular depression (as further described with respect to FIG. 4) into which an extension of the quarter-turn adapter 206 may be positioned. The user interface 112 may then be turned to be coupled to the quarter-turn adapter 206. The quarter-turn adapter 206 may be selectively coupled to the superhydrophobic expansion chamber 114 via snap fitting, press fitting, and/or another coupling method which enables quick and simple removal and attachment of the quarter-turn adapter 206 to the superhydrophobic expansion chamber 114. In this way, the user interface 112 may be selectively coupled to the superhydrophobic expansion chamber 114 of the CCU 150.

In low-g environments the relative strength of motive fluid inertia compared to resistive capillary forces is captured by the Weber number We=pU²D/σ, where p is the liquid density, σ the surface tension, and U and D the characteristic fluid velocity and length scale, respectively. In reference to urination, expelling of liquid (e.g., urine) typically begins with relatively high velocities such that We>>>1, and includes end state conditions where We<<<1. On Earth this is not a problem because urine droplets typically fall due to gravity. In low-g, however, there is no falling, and anatomy-attached urine volumes can achieve uncomfortable volumes on the order of approximately 20 mL during the late stages of urination. To address the issue of anatomy-attached urine volumes, the CCU 150 employs the superhydrophobic expansion chamber 114. The superhydrophobic expansion chamber 114 is a liquid self-propelling passage comprising a superhydrophobic passage 210 and a housing 212 which circumferentially surrounds the superhydrophobic passage 210. As further described herein, a geometry and superhydrophobic characteristics of the superhydrophobic expansion chamber 114 enable self-propelling of liquid, such as urine, by nature of the liquid interacting with the superhydrophobic material and the increasing cross-sectional width of the superhydrophobic passage 210 from a first end (e.g., where the liquid is introduced to the superhydrophobic expansion chamber 114) to a second end of the superhydrophobic expansion chamber 114 (e.g., where the superhydrophobic expansion chamber 114 is coupled to the collapsible bag 118). The housing 212 may have an elliptic cylinder shape which surrounds the superhydrophobic passage 210. The superhydrophobic passage 210 may passively propels liquid (e.g., residual urine) away from an inlet of the superhydrophobic passage 210 (e.g., in face sharing contact with the user's body).

Further detail regarding configuration of the superhydrophobic passage 210 is described with respect to FIGS. 3A, 3B, 5, and 6, and further detail regarding configuration of the housing 212 is described with respect to FIGS. 3A, 3B, 5, and 7. The superhydrophobic passage 210 may be secured inside the housing 212 using a plurality of dowel pins 214. Further, a liner retaining o-ring 216 may circumferentially surround the superhydrophobic passage 210 at the first end of the superhydrophobic expansion chamber 114 to hold together liners which form the superhydrophobic passage 210.

A vent ring 218 is configured as an annular ring with a plurality of vent holes 220 positioned radially at a first side 222 of the vent ring 218. The vent ring 218 is further configured with a cavity which may extend from a second side 224 of the vent ring 218 to the first side 222 of the vent ring 218, where the first side 222 of the vent ring has a base, such that gas flowing through the cavity may exit the vent ring 218 via the plurality of vent holes 220, as described with respect to FIG. 7. In other words, the vent ring 218 is configured as two concentric rings with a space therebetween which is sealed at the first side 222 and open at the second side 224. A superhydrophobic membrane 226 configured as an annular perforated superhydrophobic screen may be positioned in the cavity of the vent ring 218 to repel liquid from exiting the CCU 150 via the plurality of vent holes 220. An inner snap ring 228 and an inner snap ring liner 230 may be positioned in an interior of the vent ring 218. When the CCU 150 is assembled, the housing 212 of the superhydrophobic expansion chamber 114 may extend into and be circumferentially surrounded by the vent ring 218. The housing 212 may further be snap fit or press fit into the inner snap ring 228 at a second end of the superhydrophobic expansion chamber 114, opposite the first end, as further described with respect to FIGS. 3A-3B and 5-7. The outer snap ring 116 may circumferentially surround a lower region of the vent ring 218 (e.g., not including the plurality of vent holes 220) such that the plurality of vent holes 220 may fluidically couple an exterior of the CCU 150 and an interior of the CCU 150 (e.g., an interior of the collapsible bag 118) when the lid 110 is detached from the CCU 150. The outer snap ring 116 may be coupled to the vent ring 218 via a double-sided adhesive ring 232.

As shown in FIG. 2 and further described with respect to FIG. 3A, an upper region 240 of the collapsible bag 118, adjacent to an inlet 242 of the collapsible bag 118, may not include a vane structure. An extension liner 238 may be positioned in the upper region 240 of the collapsible bag 118 and have an annular configuration. When the CCU 150 is assembled, a second region 234 of the outer snap ring 116 may circumferentially surround the upper region 240 of the collapsible bag 118. Thus, the upper region 240 of the collapsible bag 118 may be positioned radially between the second region 234 of the outer snap ring 116 and the extension liner 238. The collapsible bag 118, the outer snap ring 116, and the extension liner 238 may be positioned and coupled using a bag clamp ring 246. The bag clamp ring 246 may circumferentially surround a portion of the second region 234 of the outer snap ring 116, adjacent to a first region 236 of the outer snap ring 116, and may be secured (e.g., a diameter of the bag clamp ring 246 may be decreased) by tightening a screw 248, which may be a button head hex drive screw, and a flanged fastener insert 250. Each of the superhydrophobic passage 210, the superhydrophobic membrane 226, the inner snap ring liner 230, the double-sided adhesive ring 232, and the extension liner 238 may be superhydrophobic substrates which are formed of and/or coated in superhydrophobic material.

As further described with respect to FIGS. 8-10, the collapsible bag 118 is configured with a superhydrophilic vane structure 244, which may be formed of superhydrophilic material and used to wick liquid away from the superhydrophobic expansion chamber 114. The collapsible bag 118 may also be configured with a label 252, which may be a pre-printed label and/or a writeable surface for indicating a user of the CCU 150, and a first Velcro fastener 254 and a second Velcro fastener 256 for positioning the CCU 150 during use and/or storage of the CCU 150. The collapsible bag 118 is further configured with elements for draining liquid from the CCU 150, which may include at least one drain port 264, a drain tube 260, and a pinch valve 262. The pinch valve 262 is positioned on the drain tube 260 to control drainage of liquid from the CCU 150, wherein the drain tube 260 is coupled to a barbed drain port 258 (e.g., of the at least one drain port 264), which acts as an interface between an interior of the collapsible bag 118 and the exterior of the CCU 150. In embodiments where the CCU 150 includes more than one drain port 264 positioned on the collapsible bag 118, drain ports may be located based on a desired port location, liquid location, and/or gas location. For example, additional drain ports may be used to inject and/or remove gas, and/or remove liquid.

FIG. 3A shows a side view 302, a top view 304, and a cross-sectional profile view 306 of the CCU 150 of FIGS. 1 and 2. Elements of the CCU 150 described with respect to FIGS. 1 and 2 which are included in FIG. 3A are equivalently numbered and may not be reintroduced, for brevity. An axis system 310 is provided in FIG. 3A and FIG. 3B for reference. The y-axis may be a vertical axis (e.g., parallel to a gravitational axis), the x-axis may be a lateral axis (e.g., horizontal axis), and the z-axis may be a longitudinal axis, in one example. However, the axes may have other orientations, in other examples. The CCU 150 may be used in reduced-gravity, microgravity, and/or low-gravity (low-g) environments, thus the y-axis may not be parallel to the gravitational axis and the axis system 310 is used herein to describe relative orientations of elements of the CCU 150. The CCU 150 may also be used in environments having Earth's gravity (e.g., 1-g) or any other gravity. The user interface 112, a superhydrophobic passage 210, and a superhydrophobic membrane 226 may be monolithic superhydrophobic substrates secured to the otherwise 3D printed parts (e.g., which may or may not be superhydrophobic) of the CCU 150 via adhesive. Printed lay-ups, a superhydrophilic vane, and a superhydrophilic bag liner are identified and further described with respect to FIGS. 3B and 8-10, wherein the superhydrophilic bag liner is comprised of two heat staked axial sections.

The collapsible bag 118 may be configured as a wedge which tapers to a thin end at a tapered end 314, wherein the top view 304 of the CCU 150 illustrates a tapering profile of the collapsible bag 118. As shown in the top view 304, the collapsible bag 118 has a conical configuration when viewed from the top down, where the inlet 242 has a circular opening with an inlet diameter 312 which gradually tapers to a knife-edge at a tapered end 314 of the collapsible bag 118. The knife-edge of the tapered end 314 has a negligible width, where the width is parallel to the z-axis with respect to the axis system 310.

As shown in the side view 302 and the cross-sectional profile view 306, the collapsible bag 118 has an isosceles trapezoid profile when viewed from the side. The tapered end 314 has a base length 316 which may be greater than the inlet diameter 312, in some embodiments. In the embodiments of FIG. 3A, the base length 316 is parallel with the y-axis, with respect to the axis system 310. A diameter of the collapsible bag 118 changes from a circular shape at the inlet 242 having the inlet diameter 312, to an oval shape, to the knife-edge at the tapered end 314 having the base length 316 moving along the x-axis from left to right. Thus, a length of the collapsible bag 118 which is parallel to the y-axis increases from left to right and a width of the collapsible bag which is parallel to the z-axis decreases from left to right. Further details regarding configuration of the collapsible bag 118 are described with respect to FIGS. 8-10.

Turning to FIG. 3B, an annotated profile view 308 of the CCU 150 is shown which illustrates a schematic representation of liquid collection and separation mechanisms. As briefly described above, the CCU 150 may be deployed in partial-g or low-g environments. Liquid collection and separation using the CCU 150 may simulate effects of gravity if a gravitational axis were parallel with the x-axis, with reference to the axis system 310. In this way, gravity may act in a rightward direction (e.g., from left to right). Briefly, liquid collection and separation using the CCU 150 may include passive migration of liquid (e.g., urine), including jets, droplets, and coalesced liquid to the right, and passive migration of displaced gas (e.g., air) to the left. Liquid (e.g., urine) is represented as diagonal lines in the CCU 150 and droplets within the CCU 150, and gas is represented as non-patterned circles. The superhydrophobic wetting conditions passively drive the liquid to the right, ejecting a large nearly oscillation-free droplet at a predictable velocity (e.g., into the collapsible bag 118). Superhydrophilic elements (e.g., the superhydrophilic vane structure 244) passively wick the liquid rightward along the interior corners of the collapsible bag 118, passively displacing gas bubbles leftward at a predictable rate. In this way, liquid (e.g., urine) is displaced away from a disposing source (e.g., a body) and air is wicked towards the disposing source.

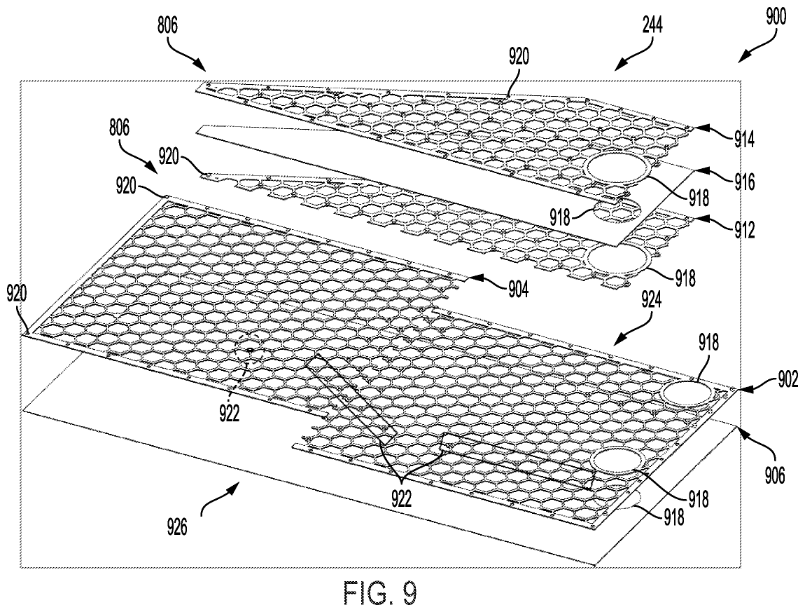

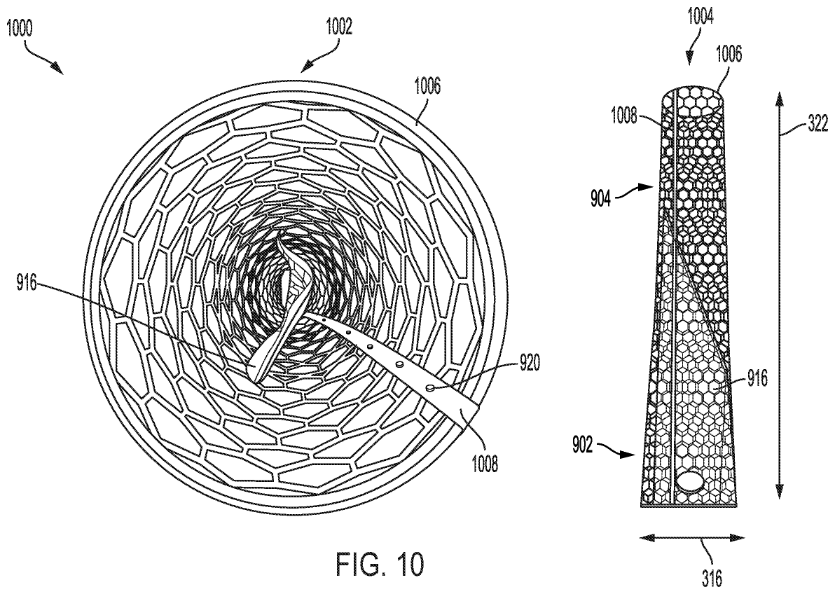

Turning to FIG. 8, elements 800 of the collapsible bag 118 are shown. The collapsible bag 118 includes a bag 802, a superhydrophilic bag liner 804, and the superhydrophilic vane structure 244, including superhydrophilic lay-up elements 806 further described with respect to FIG. 9, and a superhydrophilic vane 808. FIG. 8 also includes the label 252, the first Velcro fastener 254, the second Velcro fastener 256, the barbed drain port 258, the drain tube 260, and the pinch valve 262. As further described with respect to FIG. 10, when the collapsible bag 118 is assembled, the superhydrophilic vane 808 is positioned in between the superhydrophilic lay-up elements 806, the superhydrophilic lay-up elements 806 are positioned in the superhydrophilic bag liner 804, and the superhydrophilic bag liner 804 is positioned in the bag 802 to form the collapsible bag 118. The superhydrophilic bag liner 804 may have the same structure as the bag 802, as described with respect to FIG. 3A, and be proportionally smaller than the bag 802 in order to be positioned therein. The superhydrophilic bag liner 804 may be an example of a superhydrophilic composite bag liner. Elements 800 which are described as superhydrophilic may be formed of and/or coated with superhydrophilic materials. For example, collapsible perfectly wetting tapering polyethylene elements (e.g., the superhydrophilic bag liner 804 and superhydrophilic vane 808) may include 0.254 mm thick 70/30 Rayon/Polyester nonwoven sheets. The sheets are supported between two 0.5 mm thick largely open hexagonal-patterned PLA sheets (e.g., of the superhydrophilic vane structure 244, further described with respect to FIGS. 9-10) establishing a rigidized yet flexible composite lay-up structure of repeatable deployable geometry.

Capillary collection and containment of liquid may be significantly hampered in low-g environments for contaminated aqueous streams due to poor, widely varying, highly hysteretic, or unknown wetting conditions. The presence of superhydrophilic surfaces change this picture completely by allowing well established liquid acquisition methods of fluid control. In such cases, container geometry (e.g., of the collapsible bag 118) may be exploited to establish capillary pressure gradients within the liquid that passively drive the liquid to desired regions while simultaneously displacing gases in the form of bubbles and ullages to desired regions. In the case of the CCU 150, thin perfectly-wetting Rayon sheets in a composite lay-up structure serve as both the superhydrophilic bag liner 804 and the superhydrophilic vane structure 244 in the manner demonstrated in FIGS. 3B and 8-10. As shown in FIG. 3B, perfectly wetting liquid (e.g., urine) establishes thick films that wick ever-rightward (e.g., towards the tapered end 314), displacing bubbles and ullage ever-leftward (e.g., towards the outer snap ring 116). Separation of liquid and gas is achieved in time with the liquid contained within the superhydrophilic vane structure 244 and the collapsible bag 118, which is configured with an overall taper. For example, the superhydrophilic vane structure 244 may form an angled partition along a length of the collapsible bag 118. Containment of liquid (e.g., urine) within the CCU 150 may be stable to perturbation of the CCU 150 when the lid 110 is off. In some embodiments, the collapsible bag 118 diameter is Db=6 cm.

Turning briefly to FIG. 9, an exploded view 900 of unassembled elements of the superhydrophilic vane structure 244 is shown. Superhydrophilic lay-up elements 806 of the superhydrophilic vane structure 244 include a bottom perimeter support structure 902, a top perimeter support structure 904, perimeter wicking material 906, a top vane support structure 914, a bottom vane support structure 912, and the superhydrophilic vane 916. Each of the bottom perimeter support structure 902, the top perimeter support structure 904, the top vane support structure 914, and the bottom vane support structure 912 may have a honeycomb structure and may be formed of a flexible material which allows for bending and/or folding of the element. The perimeter wicking material 906 and the superhydrophilic vane 916 may each be a sheet of superhydrophilic material. For example, the superhydrophilic vane 916 may have a shape similar to and be proportionally smaller than that of the top vane support structure 914. Additionally, each of the top vane support structure 914, the bottom vane support structure 912, the superhydrophilic vane 916, the bottom perimeter support structure 902, and the perimeter wicking material 906 may have a circular cutout 918 (e.g., the drain port 264), which may enable positioning of elements used for drainage of the CCU 150, such as the barbed drain port 258 (shown in FIGS. 2, 3A-3B, and 8). The bottom perimeter support structure 902 may include two circular cutouts 918 to enable positioning when the superhydrophilic vane structure 244 is assembled, as described with respect to FIG. 10.

The top vane support structure 914 and the bottom perimeter support structure 902 may have fastening elements 920 disposed about a perimeter of the respective element on three sides. The bottom vane support structure 912 and the top perimeter support structure 904 may have fastening elements 920 disposed about two sides of the respective perimeter, as shown in FIG. 9. The top perimeter support structure 904 and the bottom perimeter support structure 902 may be partially coupled at an edge of each of the respective structures which does not have fastening elements. Fastening elements 920 may enable assembled elements of the superhydrophilic vane structure 244 to be disposed in the superhydrophilic bag liner 804 and ultimately in the bag 802 to form the collapsible bag 118.

For example, briefly turning to FIG. 10, perspective views 1000 of assembled elements of the superhydrophilic vane structure 244 are shown. The fastening elements 920 of the top perimeter support structure 904 may be coupled to fastening elements of the bottom perimeter support structure 902 to form an assembled perimeter support structure 1006 shown in a first perspective view 1002 and a second perspective view 1004. A structure seam 1008 along a length and a base of the assembled perimeter support structure 1006 show a position of fastening elements. As described with respect to FIG. 3A, the assembled perimeter support structure 1006 has an isosceles trapezoid configuration when viewed from the side (e.g., in the second perspective view 1004). When positioned in the collapsible bag 118, a first leg of the superhydrophilic vane structure 244 is in face sharing contact with the base length 316 of the collapsible bag 118 and a second leg of the superhydrophilic vane structure 244 is parallel and in face sharing contact with the bag length 322 of the collapsible bag 118.

Returning to FIG. 9, fastening elements 920 of the top vane support structure 914 and the bottom vane support structure 912 may enable coupling of each of the top vane support structure 914 and the bottom vane support structure 912 to themselves. For example, the superhydrophilic vane 916 may be positioned between the top vane support structure 914 and the bottom vane support structure 912, and fastening elements 920 along a first side 924 of each of the bottom vane support structure 912 and the top vane support structure 914 may be inserted into fastening elements 920 along a second side 926, opposite the first side, of the bottom vane support structure 912 and the top vane support structure 914. Face fastening elements 922 may be disposed on faces of the top perimeter support structure 904 and the bottom perimeter support structure 902 to enable coupling of the top perimeter support structure 904 and the bottom perimeter support structure 902 to the top vane support structure 914, and the bottom vane support structure 912.

Returning to FIG. 10, the superhydrophilic vane 916 may be positioned in the assembled perimeter support structure 1006 using the top vane support structure 914 and the bottom vane support structure 912. Fastening elements of the top vane support structure 914 and the bottom vane support structure 912 may be coupled to face fastening elements 922 (not shown in FIG. 10) of the top perimeter support structure 904 and the bottom perimeter support structure 902. Face fastening elements 922 may be arranged such that the top vane support structure 914, the bottom vane support structure 912, and the superhydrophilic vane 916 have an angled configuration when coupled to the assembled perimeter support structure 1006, as further described with respect to FIG. 3A.

Returning to FIG. 3A, the cross-sectional profile view 306 shows the superhydrophilic vane structure 244 arranged inside the bag 802 (as shown in detail in FIG. 8) to form the collapsible bag 118. The superhydrophilic vane 916 (e.g., supported by the top vane support structure 914 and the bottom vane support structure 912 as shown in FIG. 9) may extend a first portion 320 of a bag length 322. A cutaway view 324, taken along an A-A cut, shows an arrangement of the superhydrophilic vane 916 in the collapsible bag 118, wherein the superhydrophilic vane 916 has the angled configuration shown in FIG. 10. The angled configuration may be a herringbone or zig-zag shape, as shown in the cutaway view 324.

The cross-sectional profile view 306 further shows configurations of the user interface 112 coupled to the superhydrophobic expansion chamber 114. As described with respect to FIG. 2 and further shown in FIGS. 3A and 3B, the superhydrophobic membrane 226 configured as an annular screen is positioned between the superhydrophobic expansion chamber 114 and the collapsible bag 118. Liquid, such as urine, may be disposed into the superhydrophobic passage 210 of the superhydrophobic expansion chamber 114 via the user interface 112 and be self-propelled by the superhydrophobic passage 210 into the collapsible bag 118. The user interface 112 may be configured for use by a female user. For example, the user interface 112 may be configured as a funnel, where an inlet of the user interface 112 has a first interface diameter which gradually narrows to a second interface diameter of an outlet of the user interface 112 (e.g., where the outlet of the user interface 112 is coupled to the superhydrophobic passage 210. Further detail regarding the user interface 112 is described with respect to FIG. 4.

Turning to FIG. 4, perspective views 400 of the user interface 112 are shown. A first perspective view 402 shows the user interface 112. As briefly described with respect to FIG. 2, the user interface 112 is configured with an annular depression 418 into which an extension of the quarter-turn adapter 206 may be positioned. A second perspective view 404 shows the user interface 112 with the quarter-turn adapter 206 coupled thereto. A third perspective view 406 shows the user interface 112, including an interface inlet 408 opposite the annular depression 418 into which the quarter-turn adapter 206 is inserted. The third perspective view 406 further shows an interface outlet 412, opposite the interface inlet 408. The user interface 112 may be configured to be held against (e.g., in contact with) a female user's body for disposing urine into the user interface 112, where the interface inlet 408 is held against the user's body. For example, the user interface 112 may be configured as a funnel with an oval-like edge. As shown in a fourth perspective view 410 and a fifth perspective view 420, the interface inlet 408 of the oval-like edge may have an edge height 414 which extends a bowl height 416 of the oval-like edge, in some embodiments. Liquid (e.g., urine) may flow into the funnel of the user interface 112 and be directed into the interface inlet 408. The liquid may exit the user interface 112 via the interface outlet 412 and flow into the superhydrophobic expansion chamber coupled thereto, as shown in FIG. 3A. In some embodiments, the user interface 112 is formed of superhydrophobic material, which may assist in self-propelling of liquid through the user interface 112 (e.g., from the interface inlet 408 to the interface outlet 412).

Returning to FIG. 3A, liquid may flow from the interface outlet 412 of the user interface into the superhydrophobic expansion chamber 114, which includes the housing 212 and the superhydrophobic passage 210. As shown in FIG. 3A and further described with respect to FIG. 5, the superhydrophobic passage 210 has a bell shape which flares from a first cross-sectional width to a second cross-sectional width.

Turning to FIG. 5, elements 500 of the superhydrophobic expansion chamber 114 are shown, including the superhydrophobic passage 210 and the housing 212. The superhydrophobic passage 210 may be formed of a first liner 502 and a second liner 504, wherein each of the first liner 502 and the second liner 504 have the same configuration. The superhydrophobic passage 210 may be formed by positioning the first liner 502 and the second liner 504 in face sharing contact (e.g., a first face 506 of the first liner 502 and a second face 508 of the second liner 504) and coupling the first liner 502 and the second liner 504 using dowels (e.g., the plurality of dowel pins 214 of FIG. 2) positioned in through holes 516 of each liner. The housing 212 may be formed of a first half 512 and a second half 514 which may be coupled by press fitting, snap fitting, using dowels or other fasteners, and so on to circumferentially surround the superhydrophobic passage 210.

A configuration of the superhydrophobic passage 210 is herein described with respect to the first liner 502 and the second liner 504, however it is to be understood that a liner of the first liner 502 and the second liner 504 shows a cross-sectional profile view of the superhydrophobic passage 210 and, when assembled, the superhydrophobic passage 210 has a three-dimensional volume. The superhydrophobic passage 210 has a first end 518 and a second end 520, opposite the first end, wherein the first end 518 has a first cross-sectional width and the second end 520 has a second cross-sectional width. The second cross-sectional width is greater than the first cross-sectional width, and a cross-sectional width of the superhydrophobic passage 210 may gradually increase from the first end 518 to the second end 520, such that the superhydrophobic passage 210 has a bell shape that flares from the first cross-sectional width at the first end 518 to the second cross-sectional width at the second end 520.

Turning to FIG. 6, an embodiment of the superhydrophobic passage 210 is shown, where a configuration of the superhydrophobic passage 210 and superhydrophobic material used to form and/or coat the superhydrophobic passage 210 may enable self-propelling of liquid through the superhydrophobic passage 210 from the first end 518 to the second end 520. The embodiment shown in FIG. 6 may be the superhydrophobic passage 210 of the CCU 150, or may be implemented independently of the CCU 150 in other systems in which liquid self-propelling is desired. The liquid used in the example of FIG. 6 is water, however it is to be understood that self-propelling of liquid as described herein with respect to FIG. 6 may operate in the same way when the liquid is urine.

A plurality of images 600 of FIG. 6 show self-propelling of liquid on a planar, horizontal tabletop, which may simulate self-propelling of liquid in a low-g environment, over time. The superhydrophobic passage 210 is positioned on a left side of each image and extends a first length (e.g., 33 mm). The planar, horizontal tabletop is coated and/or formed of a superhydrophobic substrate, and a superhydrophilic substrate (e.g., Rayon sheet) is positioned on a right side of each image. Time progressed is shown in seconds in a top right corner of each of the plurality of images 600. In a first image 602 and a second image 604, a first volume of water is injected into the superhydrophobic passage 210 at the first end 518. The second image 604 shows saturation of the superhydrophobic passage 210 with the water. The water is injected at a rate of 0.08 mL/s, which is much lower than human urination rates. The water is self-propelled through the superhydrophobic passage 210 by nature of the water interacting with the superhydrophobic material and the increasing cross-sectional width of the superhydrophobic passage 210 from the first end 518 to the second end 520. In a third image 606, a bead of the water swells at the second end 520 of the superhydrophobic passage 210. In a fourth image 608, a column of the water which extends along the first length of the superhydrophobic passage 210 ruptures. Injection of the water is halted. In a fifth image 610, a droplet of water is ejected from the second end 520 of the superhydrophobic passage 210. The droplet may have an oscillating motion and may travel from the second end 520 of the superhydrophobic passage 210 across the planar, horizontal tabletop towards the Rayon sheet, as shown in the fifth image 610, a sixth image 612, and a seventh image 614. In an eighth image 616, the droplet comes in contact with and is absorbed by the superhydrophilic substrate (e.g., the Rayon sheet).

In the embodiment shown in FIG. 6, the superhydrophobic passage 210 cleanly ejects droplets rightward for a worst case low flow rate of 0.08 mL/s (analogous to a 0.08·(5/2)³≈1.25 mL/s full scale flow rate). The ejected 0.5 mL oscillating droplets glide across the superhydrophobic tabletop and come in contact with the absorbing superhydrophilic Rayon felt sheet at right. Such demonstrations confirm capillary fluidic functions of the CCU 150: injection of liquid, passive ejection away from source using a superhydrophobic expansion, and direction of the ejected droplets toward and into an absorbing superhydrophilic material that stably coalesces, transports, and retains the liquid for subsequent processing. All superhydrophobic materials remain dry during the process. Performance of the superhydrophobic passage 210 at flow rates significantly lower than the minimum female urine flow rates (as low as 10 mL/s) and well below the desired drip flow (as low as 3.2 mL/s) indicate robust opportunities for implementation of the superhydrophobic passage.

Liquid is in effect rejected from superhydrophobic surfaces while gas is rejected from superhydrophilic surfaces. Specific to the CCU design, and analogous to a rightward acceleration of gravity, FIG. 3B identifies four passive capillary fluidic methods of value to CCU design: (1) the ejection of slow-moving liquid (e.g., urine) rightward (“downward”) away from the body, (2) the rightward (“downward”) migration of urine, (3) the leftward (“upward”) migration of gas (bubbles/ullages), and the rightward (“downward”) ejection and rebounding of liquid droplets and jets.

Returning to FIGS. 3A and 3B, wicking properties of the CCU150 are herein described. Liquid disposed into the CCU 150, as indicated by a patterned “liquid in” arrow, enters the CCU 150 at the interface inlet 408 of the user interface 112. The user interface 112 funnels the liquid into the superhydrophobic expansion chamber 114, which is configured to enable liquid self-propelling away from the first end 518 of the superhydrophobic passage 210 towards the second end 520 of the superhydrophobic passage 210. Liquid droplets are ejected from the second end 520 of the superhydrophobic passage 210 and into the collapsible bag 118. The superhydrophilic bag liner 804 and superhydrophilic vane structure 244 direct liquid droplets towards the superhydrophilic vane 808, which absorbs the liquid droplets. The superhydrophilic elements passively wick the liquid rightward along the interior corners of the container passively displacing gas (e.g., bubbles) leftward at a predictable rate.

Liquid droplets are further absorbed by the perimeter wicking material 906 shown in FIG. 9. Gas displaced by absorption of liquid may move left and be displaced out of the CCU 150 via the plurality of vent holes 220 in the vent ring 218. The superhydrophobic membrane 226 positioned annularly in the vent ring 218 between the plurality of vent holes 220 and the collapsible bag 118 may repel liquid and thus prevent liquid from exiting the CCU 150 via the plurality of vent holes 220. Further detail regarding the vent ring 218 and the plurality of vent holes 220 are described with respect to FIG. 7.

Turning to FIG. 7, an embodiment of an input assembly 700 of the CCU 150 is shown. The input assembly 700 includes the user interface 112, the superhydrophobic expansion chamber 114, the quarter-turn adapter 206 (not visible in FIG. 7), the vent ring 218, the outer snap ring 116, and the bag clamp ring 246. As shown in FIG. 7, the plurality of vent holes 220 are positioned radially in the vent ring, such that the plurality of vent holes 220 are not in line with a flow direction of liquid through the CCU 150 (e.g., as described with respect to FIG. 3B). Further, the cavity of the vent ring 218 (e.g., the annular space between two concentric rings forming the vent ring 218) is sealed on a first ring side 702 and open on a second ring side 704. The superhydrophobic membrane 226 is positioned in the cavity of the vent ring 218 at the second ring side 704 (not shown in FIG. 7), which, along with positioning of the plurality of vent holes 220, may prevent liquid from entering the cavity and thus from exiting the CCU 150 via the plurality of vent holes 220. Atmospheric pressure may be maintained within the collapsible bag 118 during urine contribution because displaced air is allowed to escape through the superhydrophobic membrane 226, which may be configured as a screen mesh with 11% porosity, in some embodiments. During rough handling (e.g., shaking, turning, or other rapid movement) of the CCU 150, free satellite droplets, mother droplets, jets, geysers, and rivulets may efficiently rebound from the screen surface of the superhydrophobic membrane 226 and from interior surfaces of the user interface 112 including superhydrophobic substrates. In some embodiments, the superhydrophobic membrane 226 employs ≈0.15 mm openings on an ≈0.40 mm center-to-center spacing, for a porosity ε≈0.11, and a measured ≈510 Pa bubble point (≈1733 Pa theoretical). The screen may thus maintain a superhydrophobic liquid contact angle >150° with low hysteresis and readily rebounds liquid which comes in contact with the screen.

Following disposal of liquid into the CCU 150 and capture of the liquid by the superhydrophilic vane 808, liquid may be drained from the CCU 150. Returning to FIGS. 3A and 3B, liquid may be drained from the barbed drain port 258 via the drain tube 260 when the pinch valve 262 is opened. For example, when liquid is accumulated in the collapsible bag 118 adjacent to the tapered end 314, the pinch valve 262 may be opened, released, and/or removed from the drain tube 260. Liquid may passively flow out of the collapsible bag 118 via the drain tube 260 in some embodiments. In other embodiments, the collapsible bag 118 may be folded, rolled, or otherwise deformed to push liquid out of the collapsible bag 118 via the drain tube 260. Following removal of liquid from the CCU 150, the CCU 150 may be collapsed for storage purposes. Slow drains may be achieved that do not entrain bubbles. Fast drains may be achieved that quickly collapse the bag, draining both liquid and gas contents.

FIG. 11 shows a plurality of collapsibility options 1100 for the CCU 150. Within reason, the CCU 150 may be collapsed by flattening, folding, and or rolling. Creasing the collapsible bag 118 is tolerable and can aid in bag deployment, however broken webs within the hexagonal lay-up may occur as a result of creasing, depending on material choice. Capillary fluidic function of the CCU 150 may not be hampered by such breaks, but potential fragments in the flow increase. The CCU 150 may be collapsed using one of the options shown in FIG. 11 prior to use or following use and drainage of the CCU 150. A first collapsibility option 1102 includes laying the CCU 150 flat and expelling air from the collapsible bag 118. A second collapsibility option 1104 includes folding the collapsible bag 118. A third collapsibility option 1106 includes rolling the collapsible bag 118 about the lid 110 of the CCU 150.

FIG. 12 illustrates a method 1200 for collecting a liquid, such as urine, using the CCU 150. The CCU 150 may be used in partial-g environments, low-g environments (e.g., microgravity), and/or normal gravity environments. The CCU 150 may be used by a human user for depositing urine therein, and the CCU 150 may be configured in such a way that droplets of urine may not remain attached to the user and/or may not splash back towards the user during use of the CCU 150.

At 1202, the method 1200 includes receiving a liquid at a first end of the superhydrophobic expansion chamber. The liquid may be introduced into the first end by a user, for example, during urination. Prior to receiving liquid, the CCU 150 may be prepared (e.g., by the user). In various embodiments, the lid 110 of the CCU 150 may be removed and temporarily stored. Additionally, the user interface 112 may be secured to the first end of the superhydrophobic expansion chamber. The user interface 112 may be coupled to the superhydrophobic expansion chamber such that the interface inlet 408 may be positioned in contact with a user's body.

At 1204, the method 1200 includes propelling liquid away from the first end using the superhydrophobic expansion chamber. As described above, a geometry and superhydrophobic materials of the superhydrophobic passage of the superhydrophobic expansion chamber may enable self-propelling of liquid through the superhydrophobic passage.

At 1206, the method 1200 includes directing liquid out of a second end of the superhydrophobic expansion chamber and into the collapsible bag 118. As the collapsible bag 118 is configured with the superhydrophilic vane structure 244, liquid which exits the superhydrophobic expansion chamber and enters the collapsible bag 118 is wicked away from the second end of the superhydrophobic expansion chamber. Directing liquid into the collapsible bag 118, which includes a superhydrophilic composite bag liner (e.g., the superhydrophilic bag liner 804) and the superhydrophilic vane structure 244, further includes wicking the liquid to a tapered end of the collapsible bag using the superhydrophilic bag liner and the superhydrophilic vane structure 244.

At 1208, the method 1200 includes releasing gas from the superhydrophobic expansion chamber gas via vent holes at the second end of the superhydrophobic expansion chamber. For example, as described with respect to FIG. 3B, the gas (e.g., air bubbles) may be passively directed out of the CCU 150 when liquid wicked by the superhydrophilic vane structure 244 displaces air which may enter the collapsible bag 118 via the user interface 112 when the CCU 150 is receiving the liquid at operation 1202. As liquid moves to the right (with respect to FIG. 3B), gas may be displaced to the left. The superhydrophobic membrane 226 positioned in the vent ring 218 prevents liquid which may be adjacent to the vent ring 218 from exiting the CCU 150 via the plurality of vent holes 220 while allowing gas to pass through the superhydrophobic membrane 226 and be released.

After the liquid has been fully received into the CCU 150 (e.g., no additional liquid is introduced into the CCU 150 by the user) and the gas has been released from the CCU 150 via the vent holes 220, the method 1200 ends. The CCU 150 may be removed from face sharing contact with the body by the user. The user interface 112 may be removed from the CCU 150 and temporarily stored. The lid 110 may be recoupled to the CCU 150, as described with respect to FIG. 1, and the CCU 150 may thus be sealed from the environment. The user interface 112 may be disinfected using a wipe or other disinfecting material. The user interface 112 may be stored for later use. The CCU 150 may be drained, as described with respect to FIGS. 2-3B and 8-10, and collapsed, as described with respect to FIG. 11, prior to storage.

In this way, a CCU is provided which may allow for urine to be propelled away from the body by a superhydrophobic expansion and captured and wicked away by a tapering superhydrophilic bag liner and tapering superhydrophilic vane structure. Pressure build-up within the bag is prevented by a superhydrophobic screen that allows displaced air to escape while reflecting spurious droplets, jets, and rivulets back into the bag.

The technical effect of the urine capture device (CCU) and liquid self-propelling passage (superhydrophobic passage) described herein is the enabling of liquid collection without air flow assistance and without power.

The disclosure also provides support for a liquid self-propelling passage, comprising: a superhydrophobic passage configured to enable liquid self-propelling away from a first end of the superhydrophobic passage towards a second end of the superhydrophobic passage, opposite the first end, wherein the superhydrophobic passage has a first cross-sectional width at the first end and a second cross-sectional width at the second end, the second cross-sectional width greater than the first cross-sectional width, such that the superhydrophobic passage has a bell shape that flares from the first cross-sectional width to the second cross-sectional width. In a first example of the system, the system further comprises: a housing having an elliptic cylinder shape which surrounds the superhydrophobic passage. In a second example of the system, optionally including the first example, the superhydrophobic passage is coupled to a liquid capture system at the second end of the superhydrophobic passage and selectively coupled to a user interface at the first end of the superhydrophobic passage. In a third example of the system, optionally including one or both of the first and second examples, the superhydrophobic passage is formed of and/or coated with a non-wetting material, the non-wetting material including at least one of laser ablated polytetrafluoroethylene (PTFE), a nano-coating, and laser ablated silicone. In a fourth example of the system, optionally including one or more or each of the first through third examples, superhydrophilic surfaces and/or elements are formed of and/or coated with an absorbent wetting material, the absorbent wetting material including at least one of Rayon and Polyester.

The disclosure also provides support for a urine capture device, comprising: a superhydrophobic expansion chamber, a user interface selectively coupled to a first end of the superhydrophobic expansion chamber, and a collapsible bag coupled to a second end of the superhydrophobic expansion chamber, opposite the first end, the collapsible bag configured with a superhydrophilic vane structure. In a first example of the system, the collapsible bag is coupled to the superhydrophobic expansion chamber via a vent ring having a plurality of vent holes disposed radially around a circumference of the vent ring and extending from an exterior of the vent ring to an interior of the vent ring. In a second example of the system, optionally including the first example, the vent ring further comprises an annular perforated superhydrophobic screen. In a third example of the system, optionally including one or both of the first and second examples, the collapsible bag further comprises a superhydrophilic bag liner. In a fourth example of the system, optionally including one or more or each of the first through third examples, the collapsible bag is configured as a wedge with a circular opening at a first end, and tapers to a thin edge at a second end. In a fifth example of the system, optionally including one or more or each of the first through fourth examples, the superhydrophilic vane structure is configured as a right triangle where, when positioned in the collapsible bag, a first leg of the superhydrophilic vane structure is in face sharing contact with a tapered end of the collapsible bag and a second leg of the superhydrophilic vane structure is parallel and in face sharing contact with a bag length of the collapsible bag. In a sixth example of the system, optionally including one or more or each of the first through fifth examples, the superhydrophilic vane structure when positioned in the collapsible bag forms an angled partition along a length of the collapsible bag. In a seventh example of the system, optionally including one or more or each of the first through sixth examples, superhydrophobic elements are formed of and/or coated with a non-wetting material, such as laser ablated PTFE and/or FEP and silicone ablated PTFE and/or FEP, and superhydrophilic elements are formed of and/or coated with a wetting material, such as nonwoven Rayon/Polyester sheets. In a eighth example of the system, optionally including one or more or each of the first through seventh examples, the collapsible bag is foldable and rollable. In a ninth example of the system, optionally including one or more or each of the first through eighth examples, the collapsible bag is configured with at least one drain port.

The disclosure also provides support for a method for passively directing liquid, comprising: receiving liquid at a first end of a superhydrophobic expansion chamber, propelling the liquid away from the first end using the superhydrophobic expansion chamber, where the superhydrophobic expansion chamber enables self-propelling of the liquid from the first end to a second end of the superhydrophobic expansion chamber, directing liquid out of the second end of the superhydrophobic expansion chamber, and releasing gas from the superhydrophobic expansion chamber via a plurality of vent holes at the second end of the superhydrophobic expansion chamber. In a first example of the method, the liquid is directed away from walls of the superhydrophobic expansion chamber by the superhydrophobic material. In a second example of the method, optionally including the first example, the method is operated in a reduced- or low-gravity environment. In a third example of the method, optionally including one or both of the first and second examples, directing liquid out of the second end of the superhydrophobic expansion chamber further comprises directing the liquid into a collapsible bag comprised of a superhydrophilic composite bag liner and a superhydrophilic vane structure, and wicking the liquid to a tapered end of the collapsible bag using the superhydrophilic composite bag liner and the superhydrophilic vane structure. In a fourth example of the method, optionally including one or more or each of the first through third examples, gas from the collapsible bag is displaced by wicking of the liquid to the tapered end of the collapsible bag, and is released from the collapsible bag via an annular perforated superhydrophobic screen.

FIGS. 1-11 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, the term “approximately” is construed to mean plus or minus five percent of the range unless otherwise specified.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A liquid self-propelling passage, comprising:
a superhydrophobic passage configured to enable liquid self-propelling away from a first end of the superhydrophobic passage towards a second end of the superhydrophobic passage, opposite the first end, wherein the superhydrophobic passage has a first cross-sectional width at the first end and a second cross-sectional width at the second end, the second cross-sectional width greater than the first cross-sectional width, such that the superhydrophobic passage has a bell shape that flares from the first cross-sectional width to the second cross-sectional width.

2. The liquid self-propelling passage of claim 1, further comprising a housing having an elliptic cylinder shape which surrounds the superhydrophobic passage.

3. The liquid self-propelling passage of claim 1, wherein the superhydrophobic passage is coupled to a liquid capture system at the second end of the superhydrophobic passage and selectively coupled to a user interface at the first end of the superhydrophobic passage.

4. The liquid self-propelling passage of claim 1, wherein the superhydrophobic passage is formed of and/or coated with a non-wetting material, the non-wetting material including at least one of laser ablated polytetrafluoroethylene (PTFE), a nano-coating, and laser ablated silicone.

5. The liquid self-propelling passage of claim 1, wherein superhydrophilic surfaces and/or elements are formed of and/or coated with an absorbent wetting material, the absorbent wetting material including at least one of Rayon and Polyester.

6. A urine capture device, comprising:
a superhydrophobic expansion chamber;
a user interface selectively coupled to a first end of the superhydrophobic expansion chamber; and
a collapsible bag coupled to a second end of the superhydrophobic expansion chamber, opposite the first end, the collapsible bag configured with a superhydrophilic vane structure.

7. The urine capture device of claim 6, wherein the collapsible bag is coupled to the superhydrophobic expansion chamber via a vent ring having a plurality of vent holes disposed radially around a circumference of the vent ring and extending from an exterior of the vent ring to an interior of the vent ring.

8. The urine capture device of claim 7, wherein the vent ring further comprises an annular perforated superhydrophobic screen.

9. The urine capture device of claim 6, wherein the collapsible bag further comprises a superhydrophilic bag liner.

10. The urine capture device of claim 9, wherein the collapsible bag is configured as a wedge with a circular opening at a first end, and tapers to a thin edge at a second end.

11. The urine capture device of claim 10, wherein the superhydrophilic vane structure is configured as a right triangle where, when positioned in the collapsible bag, a first leg of the superhydrophilic vane structure is in face sharing contact with a tapered end of the collapsible bag and a second leg of the superhydrophilic vane structure is parallel and in face sharing contact with a bag length of the collapsible bag.

12. The urine capture device of claim 9, wherein the superhydrophilic vane structure when positioned in the collapsible bag forms an angled partition along a length of the collapsible bag.

13. The urine capture device of claim 9, wherein superhydrophobic elements are formed of and/or coated with a non-wetting material, such as laser ablated PTFE and/or FEP and silicone ablated PTFE and/or FEP, and superhydrophilic elements are formed of and/or coated with a wetting material, such as nonwoven Rayon/Polyester sheets.

14. The urine capture device of claim 6, wherein the collapsible bag is foldable and rollable.

15. The urine capture device of claim 6, wherein the collapsible bag is configured with at least one drain port.

16. A method for passively directing liquid, comprising:
receiving liquid at a first end of a superhydrophobic expansion chamber;
propelling the liquid away from the first end using the superhydrophobic expansion chamber, where the superhydrophobic expansion chamber enables self-propelling of the liquid from the first end to a second end of the superhydrophobic expansion chamber;
directing liquid out of the second end of the superhydrophobic expansion chamber; and
releasing gas from the superhydrophobic expansion chamber via a plurality of vent holes at the second end of the superhydrophobic expansion chamber.

17. The method of claim 16, wherein the liquid is directed away from walls of the superhydrophobic expansion chamber by the superhydrophobic material.

18. The method of claim 16, wherein the method is operated in a reduced- or low-gravity environment.

19. The method of claim 16, wherein directing liquid out of the second end of the superhydrophobic expansion chamber further comprises directing the liquid into a collapsible bag comprised of a superhydrophilic composite bag liner and a superhydrophilic vane structure, and wicking the liquid to a tapered end of the collapsible bag using the superhydrophilic composite bag liner and the superhydrophilic vane structure.

20. The method of claim 19, wherein gas from the collapsible bag is displaced by wicking of the liquid to the tapered end of the collapsible bag, and is released from the collapsible bag via an annular perforated superhydrophobic screen.
